(19) United States
(12) Patent Application Publication
(10) Pub. No.: US 2024/0091010 A1
(43) Pub. Date: Mar. 21, 2024

(54) VALVE CLAMP FOR PREVENTING LEAFLET INJURY, VALVE CLAMPING SYSTEM, AND METHOD FOR REPAIRING VALVE

(71) Applicant: HANGZHOU VALGEN MEDTECH CO., LTD., Hangzhou, Zhejing (CN)

(72) Inventors: Xianzhang ZHENG, Hangzhou (CN); Tingchao ZHANG, Hangzhou (CN); Weiwei ZHANG, Hangzhou (CN)

(73) Assignee: HANGZHOU VALGEN MEDTECH CO., LTD., Hangzhou (CN)

(21) Appl. No.: 18/256,651

(22) PCT Filed: Dec. 22, 2021
(86) PCT No.: PCT/CN2021/140454

§ 371 Date: Jun. 9, 2023

(30) Foreign Application Priority Data
Dec. 28, 2020 (CN) 202011583358.3
Dec. 28, 2020 (CN) 202023226904.6

(51) Int. Cl. A61F 2/24

(52) U.S. Cl. CPC A61F 2/246; A61F 2/2436

(57) ABSTRACT

M Disclosed is a valve clamp for preventing leaflet injury, a valve clamping system, and a method for repairing a valve. The valve clamp for preventing leaflet injury comprises a fixed base and a pair of clamp arms that are openably and closeably disposed on opposite sides of the fixed base, wherein each of the clamp arms has a connection end connected to the fixed base, a free end opposite to the connection end, and a gripping section between the connection end and the free end, the gripping section is at least partially recessed inwardly to form a capture zone, and the capture zone extends toward the free end and then extends toward a direction away from the capture zone of the other clamp arm to form a first flanging section.

### CROSS-REFERENCE TO RELATED APPLICATIONS

The present disclosure claims all the benefits of the Chinese patent application No. 202011583358.3, filed on Dec. 28, 2020 before the China National Intellectual Property Administration of the People's Republic of China, entitled “Valve Clamp For Preventing Leaflet Injury And Valve Clamping System”, and the Chinese Utility Model application No. 202023226904.6, filed on Dec. 28, 2020 before the China National Intellectual Property Administration of the People's Republic of China, entitled “Valve Clamp For Preventing Leaflet Injury And Valve Clamping System”, which are explicitly incorporated herein by reference in their entirety.

### FIELD

The present disclosure generally relates to a field of medical instruments, particularly to a valve clamp for preventing leaflet injury, a valve clamping system, and a method for repairing a valve.

### BACKGROUND

A mitral valve is a one-way valve located between the left atrium and the left ventricle of a heart. A normal and healthy mitral valve can control blood to flow from the left atrium to the left ventricle while avoiding blood flowing from the left ventricle to the left atrium. The mitral valve comprises a pair of leaflets, called an anterior leaflet and a posterior leaflet respectively. When edges of the anterior and posterior leaflets are coapted, the mitral valve can fully close, preventing blood from flowing from the left ventricle to the left atrium. When the leaflet or related structure of the mitral valve organically changes or functionally changes, the anterior leaflet and the posterior leaflet of the mitral valve are poorly coapted. Thus, when the left ventricle contracts, the mitral valve cannot fully close, causing blood to regurgitate from the left ventricle to the left atrium, thereby leading to a series of pathophysiological changes referred to as “mitral valve regurgitation”.

There is a minimally invasive treatment instrument that delivers a valve clamp through an interventional catheter to the mitral valve and grips the anterior and posterior leaflets of the mitral valve simultaneously by relative opening and closing of the clamp based on the edge-to-edge surgery principle of the valve, thereby achieving the goal of fixing the leaflet and reducing mitral valve regurgitation.

### SUMMARY

In an aspect, the present disclosure relates to a valve clamp for preventing leaflet injury comprising a fixed base and a pair of clamp arms that are openably and closeably disposed on opposite sides of the fixed base, wherein each of the clamp arms has a connection end connected to the fixed base, a free end opposite to the connection end, and a gripping section between the connection end and the free end, the gripping section is at least partially recessed inwardly to form a capture zone, and the capture zone extends toward the free end and then extends toward a direction away from the capture zone of the other clamp arm to form a first flanging section.

In some embodiments, an included angle A ranging from 90° to 150° is formed between a tangent of an end of the first flanging section and an axial direction of the gripping section.

In some embodiments, an included angle B ranging from 20° to 60° is formed between axial directions of the two gripping sections after the pair of clamp arms close relative to the fixed base.

In some embodiments, an included angle C ranging from −30° to 10° is formed between an extending line of the first flanging section and a line perpendicular to a central axis of the valve clamp.

In some embodiments, a half of the angle B plus 90° is equal to a sum of the angle A and the angle C.

In some embodiments, an anti-slipping portion is provided between the capture zone and the first flanging section, and a depth of the anti-slipping portion is less than a depth of the capture zone.

In some embodiments, the clamp arm further comprises a pair of second flanging sections, and each of the second flanging sections is formed by extending from the capture zone to each side and is smoothly connected to each side of the first flanging section.

In some embodiments, the clamp arm further comprises a pair of third flanging sections, and each of the third flanging sections is formed by extending outward from each side of the capture zone and is smoothly connected to the second flanging section on the same side thereof.

In some embodiments, the third flanging section sequentially comprises the following from a distal end to a proximal end: a first part; a second part; and a third part, wherein a transverse dimension of the first part gradually increases from the distal end toward the proximal end to be substantially equal to a transverse dimension of the second part, the transverse dimension of the second part decreases gradually from the distal end toward the proximal end to be substantially equal to a transverse dimension of the third part.

In some embodiments, the clamp arm further comprises a pair of fourth flanging sections, and each of the fourth flanging sections is formed by extending proximally from the third flanging section on the same side thereof respectively.

In some embodiments, the clamp arm is provided with a hole for assembly, the fourth flanging section is provided next to the hole, and the fourth flanging section has an axial dimension larger than a diameter of the hole.

In some embodiments, the valve clamp for preventing leaflet injury further comprises a pair of gripping arms, wherein each of the gripping arms is disposed between the fixed base and the clamp arm, and the gripping arm is at least partially received in the inner wall of the capture zone in a natural state.

In another aspect, the present disclosure relates to a valve clamping system comprising the valve clamp for preventing leaflet injury of the present disclosure and a drive component comprising:
a drive shaft penetrating the fixed base;
a connecting seat disposed at a distal end of the drive shaft; and
a pair of connecting rods pivotably connected to both sides of the connecting seat,
wherein an end of the connecting rod is connected to the clamp arm, and
the drive shaft slides axially relative to the fixed base to drive the clamp arm to open and close relative to the fixed base by rotation of the connecting rod.

In some embodiments, the connecting seat is fixedly connected to the distal end of the drive shaft, the connecting seat is symmetrically provided with two pairs of connecting holes penetrating each other, and an end of each of the connecting rods is rotatably connected to one pair of the connecting holes.

In some embodiments, after the pair of clamp arms close relative to the fixed base, a distance between the two clamp arms and a center of rotation of the connecting rod ranges from 2.4 mm to 4 mm.

In some embodiments, when the pair of clamp arms open to an everted state relative to the fixed base, an everted angle between the two clamp arms ranges from 260° to 300°; and a span of ends of the two clamp arms ranges from 14 mm to 18 mm.

In some embodiments, when the pair of clamp arms open to an everted state relative to the fixed base, a distance between positions where each of the clamp arms abuts the connecting rod ranges from 3.6 mm to 4.4 mm.

In some embodiments, the valve clamping system further comprises a delivery component that comprises an outer sheath and a mandrel movably penetrated in the outer sheath, wherein a distal end of the mandrel is detachably connected to a proximal end of the drive shaft.

In another aspect, the present disclosure relates to a method for repairing a valve, comprising:
pushing a drive shaft and the valve clamp connected thereto from an atrium of a subject in need thereof via a valve to a ventricle by a delivery component;
adjusting the valve clamp to approach leaflets, i.e., an anterior leaflet and a posterior leaflet of the valve;
pulling a mandrel and the drive shaft proximally, driving the clamp arm to open relative to the fixed base, and adjusting the direction of the clamp arm, so that the clamp arm is perpendicular to a coaptation line of the valve;
withdrawing the entire valve clamp proximally, so that the clamp arm holds the leaflet on the ventricle side;
loosening a control wire to release the gripping arms on both sides, wherein the gripping arm on each side presses the leaflet on the atrium side and fixes the leaflet in cooperation with the clamp arm on the side to realize complete clamping of the leaflet;
pushing the mandrel and the drive shaft distally to drive the clamp arms to close when the anterior leaflet and the posterior leaflet of the valve are clamped between the pair of clamp arms and gripping arms respectively; and
releasing connection between the mandrel and the drive shaft and withdrawing the mandrel, so that the valve clamp is disconnected from the delivery component, and then withdrawing the delivery component from a body, during which the valve clamp pulls the anterior leaflet and the posterior leaflet of the valve toward each other to obtain a double orifice structure and complete edge-to-edge repair of the valve.

In some embodiments, the subject may be a human.

In some embodiments, the exemplary examples of the valve that can be used in the present disclosure includes, but is not limited to, mitral valve and tricuspid valve.

The valve clamp for preventing leaflet injury, the valve clamping system, and the method for repairing a valve in some embodiments of the present disclosure at least have the following beneficial effects: a first flanging section is disposed at an end of a clamp arm, which can cause the first flanging section to form a fitting structure with a leaflet after the leaflet enters a capture zone, thereby increasing the contact area with the leaflet, avoiding local force concentration of the leaflet, effectively reducing injury to the leaflet caused by repeated friction between an edge of the clamp arm and the leaflet as the heart beats, and reducing surgical complications.

### BRIEF DESCRIPTION OF THE DRAWINGS

The drawings herein are incorporated into the specification and constitute a part of the specification. The drawings show examples conforming to the present disclosure and are used together with the specification to explain the principle of the present disclosure.

In order to explain the technical solutions more clearly in the examples of the present disclosure or the prior art, the drawings used in the examples or the description of the prior art are briefly explained. Obviously, one skilled in the art can obtain other drawings based on these drawings without involving creative efforts.

FIG. 1 shows a front view of a valve clamping system when opened according to an exemplary embodiment.

FIG. 2 shows an oblique view of a valve clamping system when opened according to an exemplary embodiment.

FIG. 3 shows a front view of a valve clamping system when slightly opened according to an exemplary embodiment.

FIG. 4 shows an oblique view of a valve clamping system when slightly opened according to an exemplary embodiment.

FIG. 5 shows a front view of a valve clamping system when closed according to an exemplary embodiment.

FIG. 6 shows an oblique view of a valve clamping system when closed according to an exemplary embodiment.

FIG. 7 shows an oblique view of a clamp arm according to an exemplary embodiment.

FIG. 8 shows an oblique view from another angle of a clamp arm according to an exemplary embodiment.

FIG. 9 shows a top view of a clamp arm according to an exemplary embodiment.

FIG. 10 shows a front view of a clamp arm according to an exemplary embodiment.

FIG. 11 shows a right view of a clamp arm according to an exemplary embodiment.

FIG. 12 shows a right view of a clamp arm according to an exemplary embodiment on which a leaflet is lapped.

FIG. 13 shows a front view of a valve clamping system when initially clamping leaflets according to an exemplary embodiment.

FIG. 14 shows a front view of a valve clamping system after clamping leaflets according to an exemplary embodiment.

FIG. 15 shows an oblique view of a valve clamping system after a fixed base and gripping arms are removed according to an exemplary embodiment.

FIG. 16 shows an exploded view of a rotary pin according to an exemplary embodiment.

FIG. 17 shows a top assembly view of two clamp arms according to an exemplary embodiment.

FIG. 18 shows a structural parameter view of a valve clamping system when closed according to an exemplary embodiment.

FIG. 19 shows a structural parameter view of a valve clamping system when opened to a maximum angle according to an exemplary embodiment.

FIG. 20 shows a structural schematic view of a fixed base according to an exemplary embodiment.

FIG. 21 shows an assembly schematic view of a fixing member and a fixed base according to an exemplary embodiment.

FIG. 22 shows a structural schematic view of a valve clamping system and an adjustment portion according to an exemplary embodiment.

FIG. 23 shows a structural schematic view of a valve clamping system reaching leaflets according to an exemplary embodiment.

FIG. 24 shows a structural schematic view of a valve clamping system opened and preparing to clamp leaflets according to an exemplary embodiment.

FIG. 25 shows a structural schematic view of clamp arms of a valve clamping system fitting with leaflets according to an exemplary embodiment.

FIG. 26 shows a structural schematic view of clamp arms of a valve clamping system clamping leaflets according to an exemplary embodiment.

FIG. 27 shows a structural schematic view of a valve clamping system completing edge-to-edge repair of a mitral valve according to an exemplary embodiment.

### DETAILED DESCRIPTION

To make the objects, technical solutions, and advantages clearer, the technical solutions of the present disclosure are described below clearly and completely with reference to the drawings. Obviously, the described are only part of examples of the present disclosure, rather than all of the examples. Based on the examples in the present disclosure, all other examples obtained by one skilled in the art without involving inventive work fall within the protection scope of the present disclosure.

In the present disclosure, an orientation or position relationship indicated by “front”, “back”, “upper”, “lower”, “left”, “right”, “longitudinal”, “transverse”, “vertical”, “horizontal”, “top”, “bottom”, “inner”, “outer”, “head”, “tail”, and the like is an orientation or position relationship shown in the drawings, and construction and operation in a specific orientation is merely for convenience of describing the present technical solutions, rather than indicating that the device or element referred to must have a specific orientation, and thus should not be construed as limiting the present disclosure.

In the present disclosure, terms such as “mount”, “connected to”, “connect”, “fix”, “dispose”, and the like should be understood broadly unless expressly specified or defined. For example, it may be a fixed connection, a detachable connection, or an integration; it may be a direct connection, an indirect connection by an intermediary, or an internal communication or interaction relationship between two elements. When an element is referred to as being “above” or “under” another element, the element may be “directly” or “indirectly” on another element, or there may be one or more intervening elements. Specific meanings of the above terms in the present disclosure can be understood by one skilled in the art in light of specific circumstances.

In the specification of the present disclosure, unless otherwise specified, a proximal end refers to an end of an instrument or component near an operator, a distal end refers to an end of the instrument or component away from the operator; and an axial direction refers to a direction parallel to a center line between the distal end and the proximal end of the instrument or component, a radial direction refers to a direction perpendicular to the axial direction, and a circumferential direction refers to a direction around the axial direction.

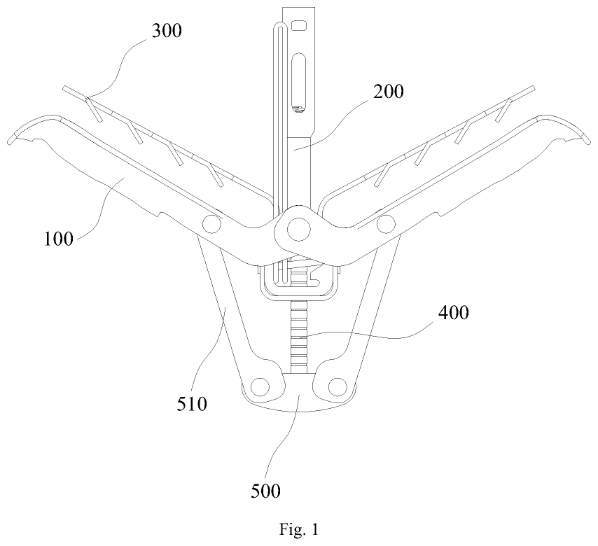

Referring to FIG. 1 to FIG. 6, a valve clamp for preventing leaflet injury of the present disclosure is transcatheterly or transapically delivered to a mitral valve of a patient through a delivery component, and a proximal end of the valve clamp is releasably connected to the delivery component. An operator remotely operates the valve clamp, grips the anterior and posterior leaflets of the mitral valve together, and then releases the connection between the delivery component and the valve clamp, thereby implanting the valve clamp within the patient's body, fixing the anterior and posterior leaflets of the mitral valve together, and achieving “edge-to-edge repair” of the mitral valve.

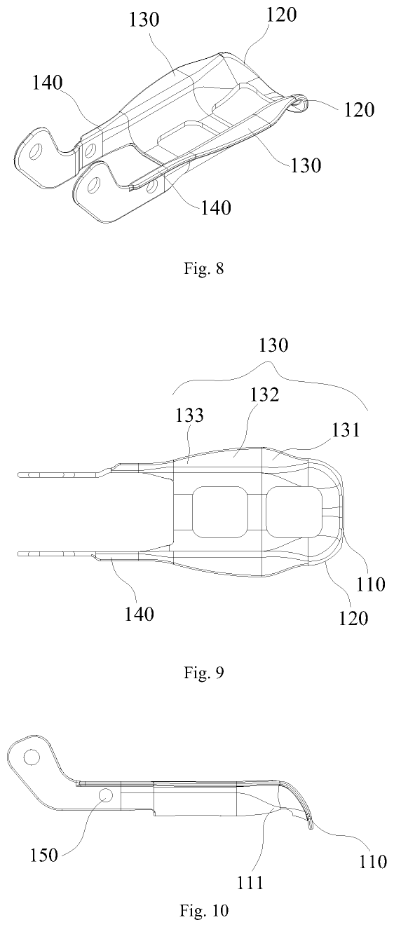

Referring to FIG. 7 to FIG. 12, the valve clamp of the present disclosure comprises a fixed base 200 and a pair of clamp arms 100 that are openably and closeably disposed on opposite sides of the fixed base 200. Each clamp arm 100 has a connection end connected to the fixed base 200, a free end opposite to the connection end, and a gripping section between the connection end and the free end. The gripping section is at least partially recessed inwardly to form a capture zone, the capture zone extends toward the free end and then extends toward a direction away from the capture zone of the other clamp arm 100 to form a first flanging section 110. The first flanging section 110 is provided at the end of the clamp arm 100, so that the leaflet 700 and the first flanging section 110 form a fitting structure, which increases a contact area and avoids local force concentration of the leaflet 700, thereby overcoming the defect that the clamp arm of the valve clamp has a low degree of fit with the leaflet 700 and easily causes injury to the leaflet 700 in the prior art.

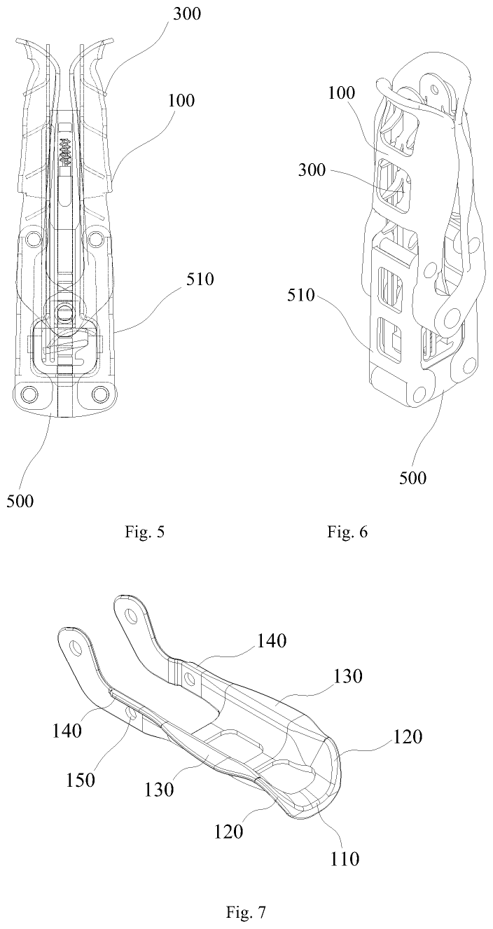

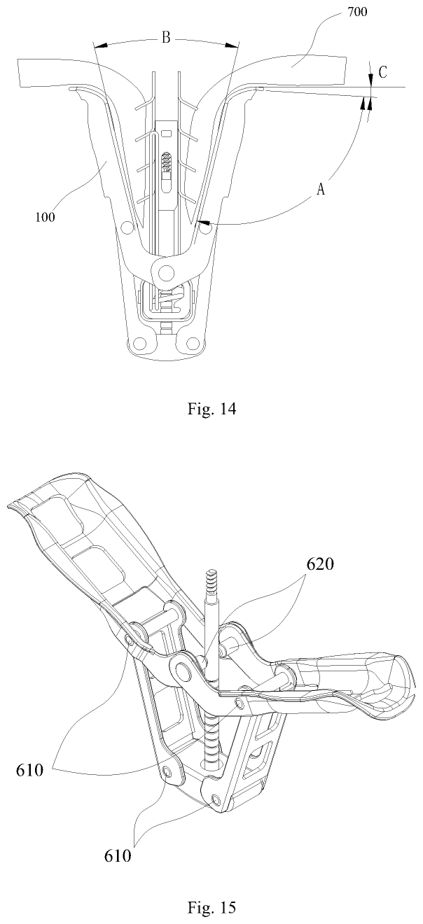

In the technical solutions of the present disclosure, the gripping section of the clamp arm 100 is a main force zone used to grip the leaflet 700, and the surface of the gripping section is recessed inwardly and is provided with the first flanging section 110, thereby achieving the following beneficial effects in different usage states: firstly, referring to FIG. 5 and FIG. 6, in a delivery state, the gripping arm 300 is at least partially received in the inner surface of the clamp arm 100, which reduces the outer diameter of the valve clamp and facilitates delivery; secondly, referring to FIG. 13 and FIG. 14, after the clamp arm 100 cooperates with the gripping arm 300 to clamp the leaflet 700, the recessed inner surface can increase the contact area between the clamp arm 100 and the leaflet 700 and cause the gripping arm 300 to press the leaflet 700 into the inner surface of the clamp arm 100, which increases the gripping force; and the first flanging section 110 is disposed at the end of the clamp arm 100, which can cause the first flanging section 110 to form a fitting structure with the leaflet 700 after the leaflet 700 enters the capture zone, thereby increasing the contact area with the leaflet 700, avoiding local force concentration of the leaflet 700, reducing injury of the leaflet caused by repeated friction between the edge of the clamp arm 100 and the leaflet 700 as the heart beats, and reducing surgical complications.

In some embodiments, the valve clamp further comprises a pair of gripping arms 300, each gripping arm 300 is disposed between the fixed base 200 and the clamp arm 100, and the gripping arm 300 is at least partially received in the inner wall of the capture zone in a natural state. When the gripping arm 300 is released, the gripping arm 300 moves close to the clamp arm 100 and cooperates with the clamp arm 100 to clamp the tissue of the leaflet 700 located therebetween. The gripping arm 300 is received in the capture zone in a natural state, which can reduce the overall size of the valve clamp, and can play a protective role on the gripping arm 300 during delivery of the valve clamp, thereby preventing injury to the heart tissue by the gripping arm 300.

In some embodiments, in order to increase biocompatibility between the valve clamp and the leaflet and facilitate crawling of endothelial cells, a biocompatible polymer film selected from materials such as PET and PTFE may be applied to a gripping surface of the clamp arm 100 (i.e., a surface facing the gripping arm 300) and a gripping surface of the gripping arm 300 (i.e., a surface facing the clamp arm 100), and the materials of the film applied to the clamp arm 100 and the gripping arm 300 may be the same or different.

In some embodiments, in order to increase the gripping force of the gripping arm 300 and the clamp arm 100 to the leaflet 700, at least one row of barbs may be provided on a surface of the gripping arm 300 facing the clamp arm 100 to prevent the leaflet 700 from slipping from the inner surface of the clamp arm 100.

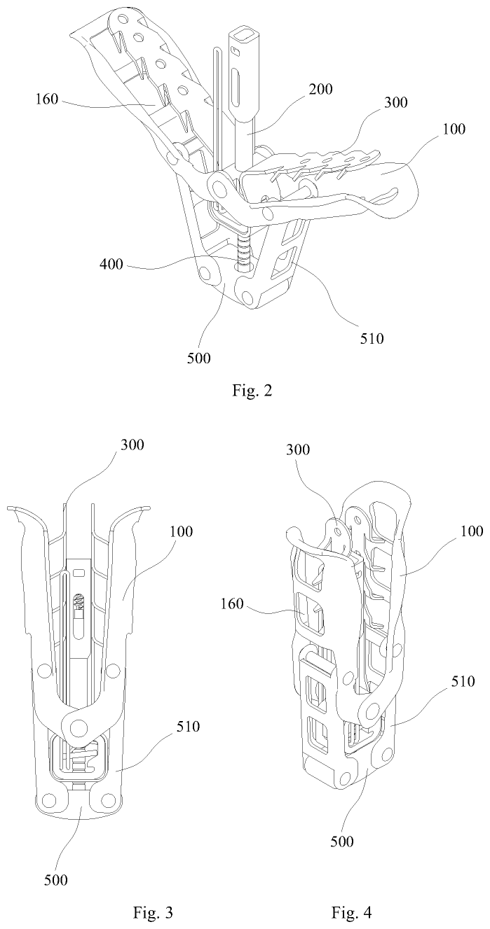

Referring to FIG. 2 and FIG. 4, the gripping section of the clamp arm 100 is further provided with at least one opening 160 penetrating the surface of the gripping section, which means: (1) reducing weight of the clamp arm 100 and lightening the movement load applied to the leaflet 700 by the clamp arm 100; (2) when a polymer film is applied to the surface of the clamp arm 100 by way of sewing, facilitating fixation of a suture, increasing the attachment strength of the film, and preventing the film from being offset during motion of the leaflet 700; (3) facilitating crawling of endothelial cells; and (4) and in a delivery state, both of the gripping arm 300 and the clamp arm 100 rest on the outside of the fixed base 200, during which barbs on the top of the gripping arm 300 are at least partially received or pressed into the openings 160 of the clamp arm 100, but both of them are not stuck, thereby reducing the outer diameter of the valve clamp and facilitating delivery.

In some embodiments, referring to FIG. 14, an included angle A ranging from 90° to 150° is formed between the tangent of the end of the first flanging section 110 and the axial direction of the gripping section. When the leaflet 700 is disposed inside the clamp arm 100, a part of the leaflet 700 is disposed in the capture zone, and a part of the leaflet 700 disposed outside the clamp arm 100 rest on the first flanging section 110. The size of the included angle A determines the resting form of the leaflet 700, and an angle range of 90° to 150° can provide good support for the leaflet 700 while preventing the leaflet 700 from being injured due to excessive force concentration.

In some embodiments, after the pair of clamp arms 100 are closed relative to the fixed base 200, an included angle B ranging from 20° to 60° is formed between the axial directions of the two gripping sections. The size of the angle B is related to the effect of regurgitation treatment. The smaller the angle B, the better the coaptation effect of the two leaflets 700 after the clamp arm 100 closes, the more obvious the regurgitation reduction effect, but correspondingly a pull stress on the leaflet 700 increases. When the angle B is too large, the leaflets 700 cannot be coapted well, thereby not effectively reducing regurgitation, so the included angle B ranges from 20° to 60°.

In some embodiments, an included angle C ranging from −30° to 10° is formed between the extension line of the first flanging section 110 and the line perpendicular to the central axis of the valve clamp. The angle C is a direction angle and has a direction specification. In the present disclosure, it is specified that an angle above the perpendicular line is negative and an angle below the perpendicular line is positive; the size of the angle C is related to the fitting effect of the leaflet 700 with the first flanging section 110, the larger the negative angle, the better the fitting effect, but the corresponding stress also increases, which increases the risk of injuring the leaflet 700; and the larger the positive angle, the worse the fitting effect, so the included angle C ranges from −30° to 10°.

In some embodiments, B/2+90°=A+C. The size of the angle A is related to the angle C. The larger the angle A, the larger the negative angle of the angle C, thereby increasing the risk of injuring the leaflet 700; the smaller the angle A, the larger the positive angle of the angle C, thereby leading to poor fitting effect. Therefore, the range of the included angle A should be set at 90° to 150°, and 95° to 120° in some embodiments.

In some embodiments, referring to FIG. 10, an anti-slipping portion 111 is provided between the capture zone and the first flanging section 110, and the depth of the anti-slipping portion 111 is less than the depth of the capture zone. The depth herein refers to a longitudinal depth generated along the radial direction of the gripping section, wherein the depth of the capture zone determines the receiving effect of the leaflet. The anti-slipping portion 111 may be provided as a smooth transitional shape such as a cambered surface or a curved surface with varying curvature, and the anti-slipping portion 111 has a limiting effect on the leaflet 700 disposed on the gripping section, which prevents the leaflet 700 from slipping from the capture zone.

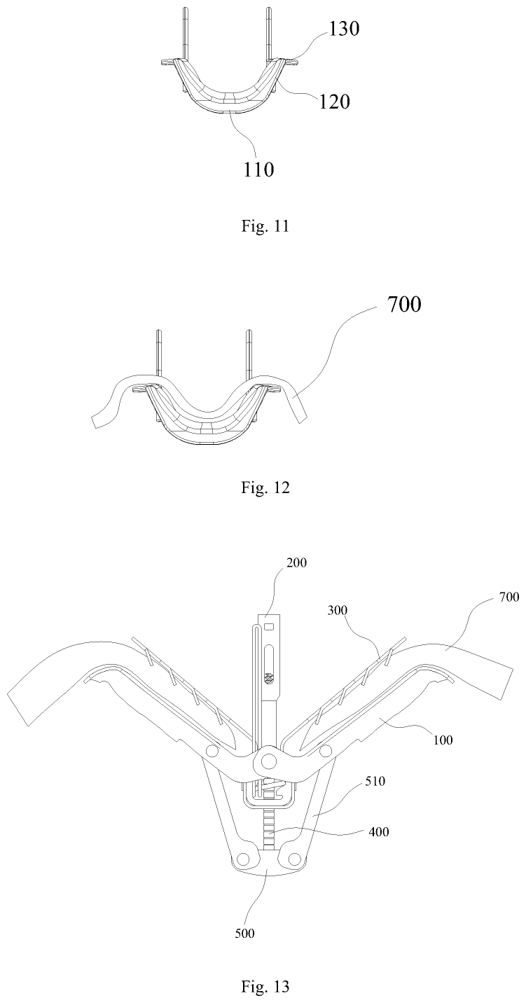

In some embodiments, referring to FIG. 11 and FIG. 12, the clamp arm 100 further comprises a pair of second flanging sections 120, each second flanging section 120 is formed by extending from the capture zone to both sides respectively, and each second flanging section 120 is smoothly connected to each side of the first flanging section 110. The second flanging section 120 of the clamp arm 100 is located at a transitional position from the gripping section to the end of the clamp arm 100. At this position, the second flanging section 120 is formed by firstly closing up from the bottom to the centerline of the gripping section of the clamp arm 100 and then expanding upward and gradually outward, which means that: (1) the second flanging section 120 can increase the area of the expanding flanging at the upper part, and increase the contact area between the leaflet 700 and the clamp arm 100 without increasing size; and (2) the second flanging section 120 does not need to additionally increase the overall size of the valve clamp, which facilitates reducing the size of the delivery component. In some embodiments, in order to achieve smooth connection between the second flanging section 120 and the first flanging section 110, the second flanging section 120 has a cambered transition zone for reducing bending stress of the leaflet 700.

In some embodiments, referring to FIG. 9, the clamp arm 100 further comprises a pair of third flanging sections 130. Each third flanging section 130 is formed by extending outward from each side of the capture zone, and each third flanging section 130 is smoothly connected to the second flanging section 120 on the same side thereof. Since the leaflet 700 is pressed into the inner surface of the clamp arm 100, there is a certain pulling force between the leaflet 700 and the edge of the clamp arm 100, while the third flanging section 130 can increase the stressed area of the capture zone, preventing the leaflet 700 from stress injury caused by the edge of the clamp arm 100. The third flanging section 130 is smoothly connected to the second flanging section 120, so that the position with which the leaflet 700 may be in contact remains smooth, avoiding injury to the leaflet 700. In some embodiments, the shape of the third flanging section 130 may be a parallel and equal-width structure, or may be an unequal-width structure that transitions smoothly through a curve.

In some embodiments, referring to FIG. 9, the third flanging section 130 comprises the following sequentially from the distal end to the proximal end: a first part 131; a second part 132; and a third part 133, wherein the transverse dimension of the first part 131 gradually increases from the distal end toward the proximal end to be substantially equal to the transverse dimension of the second part 132, and the transverse dimension of the second part 132 decreases gradually from the distal end toward the proximal end to be substantially equal to the transverse dimension of the third part 133.

In some embodiments, referring to FIG. 7 to FIG. 9, the clamp arm 100 further comprises a pair of fourth flanging sections 140, each being formed by extending from the third flanging section 130 on the same side thereof to the proximal end. The fourth flanging section 140 of the clamp arm 100 is located at the proximal end of the clamp arm 100 near the mounting position of the fixed base 200. Effects of the fourth flanging section 140 are: on one hand, increasing the contact area between the clamp arm 100 and the leaflet 700 to prevent the clamp arm 100 from scratching the leaflet 700; on the other hand, acting as a reinforcing rib to prevent deformation of the proximal end of the clamp arm 100.

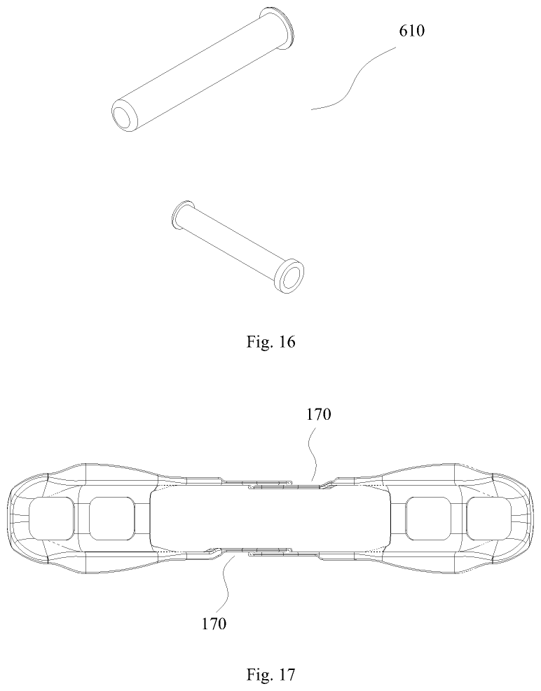

In some embodiments, referring to FIG. 9 and FIG. 10, the clamp arm 100 is provided with a hole 150 for assembly, the fourth flanging section 140 is provided next to the hole 150, and the fourth flanging section 140 has an axial dimension larger than the diameter of the hole 150. The hole 150 herein is used to rotatably connect other components, for example, as a pin hole, in which a clamp arm pin is disposed, referring to FIG. 15 and FIG. 16. Since the pulling force of the leaflet 700 is applied to the clamp arm 100 and in turn transmits to the pin of the clamp arm 100 after the valve clamp clamps the leaflet 700, the pin hole needs to withstand a large load. In some embodiments, the fourth flanging section 140 of the clamp arm 100 is used as a reinforcing rib to cause the border of the clamp arm 100 to cover the pin hole 150 in the projection direction, which effectively enhances the strength of the hole 150, thereby preventing the clamp arm 100 from failing due to insufficient strength, enhancing the strength of the clamp arm 100 itself, and ensuring the fatigue resistance of the valve clamp.

In some embodiments, referring to FIG. 17, the leg of the clamp arm 100 can be bent inward or outward to form a bending portion 170. The two bent clamp arms 100 are rotationally symmetric about the central axis, i.e., the structures of both of the clamp arms 100 on the valve clamp are identical, which not only can make the structure simple and reliable, but also can save the material cost and ensure that the center of mass of the valve clamp is in a middle position, preventing the valve clamp from being offset in the delivery process and after implantation.

The gripping arm 300 is disposed between the fixed base 200 and the clamp arm 100 for cooperating with the clamp arm 100 to clamp the tissue of the leaflet 700 located therebetween. In some embodiments, in order to ensure that the gripping arm 300 can actively approach the clamp arm 100 to press the leaflet 700 toward the clamp arm 100, the gripping arm 300 is at least partially made of a resilient material having a shape memory function and subjected to heat setting treatment, and has a natural state and a delivery state in which the gripping arm 300 can be compressed or stretched into a small sheath for delivery. In the natural state, both sides of the gripping arm 300 extend outward relative to the fixed base 200. In some embodiments, both sides of the gripping arm 300 extend proximally to facilitate cooperation with the clamp arm 100 to clamp the tissue of the leaflet 700. In some embodiments, the included angle between the two gripping arms 300 in the naturally expended state should be slightly greater than the included angle between the two clamp arms 100, i.e., the included angle between the length direction of the gripping arm 300 and the axial direction of the fixed base 200 is greater than or equal to the angle between the clamp arm 100 and the axial direction of the fixed base 200 when the clamp arm 100 corresponding to the side is fully expended relative to the fixed base 200, so that the free end of each gripping arm 300 and the corresponding clamp arm 100 approach each other and have a gripping force to provide a more stable gripping force. In some embodiments, the included angle between the length direction of the gripping arm 300 and the axial direction of the fixed base 200 ranges from 0° to 150°, i.e., in the natural state, the included angle between the two gripping arms 300 can be up to 300°, and 160° to 200° in some embodiments.

In some embodiments, the free end of the gripping arm 300 is provided with a control wire hole for connecting a control wire (not shown in the drawings) of the delivery components, and the free end of the gripping arm 300 can be controlled by a control wire extending outside the patient's body. In the delivery state, the free end of the gripping arm 300 is tensioned by the control wire and fits to the surface of the fixed base 200; and upon release of control of the control wire on the free end, the gripping arm 300 is released, the gripping arm 300 recovers the natural state due to elastic shape memory properties thereof, and presses the leaflet 700 toward the clamp arm 100.

The present disclosure further discloses a valve clamping system comprising the valve clamp for preventing leaflet injury with the structure described in the present disclosure and a drive component comprising a drive shaft 400, a connecting seat 500, and a pair of connecting rods 510. The drive shaft 400 penetrates the fixed base 200, the connecting seat 500 is provided at the distal end of the drive shaft 400, a pair of connecting rods 510 are pivotably connected to both sides of the connecting seat 500, and an end of the connecting rod 510 is connected to the clamp arm 100. The drive shaft 400 slides axially relative to the fixed base 200 to drive the clamp arm 100 to open and close relative to the fixed base 200 by rotation of the connecting rod 510.

In some embodiments, an end of each connecting rod 510 is connected to the clamp arm 100, and the other end is pivotably connected to the connecting seat 500, i.e., each of the clamp arms 100 is rotatably connected to the distal end of the connecting seat 500 of the drive shaft 400 by the connecting rod 510 on the corresponding side. The drive shaft 400 movably penetrates the fixed base 200, and the connecting rod 510 rotates and drives the clamp arm 100 to open and close relative to the fixed base 200 as the drive shaft 400 slides axially relative to the fixed base 200. In some embodiments, an end of a connecting section of each clamp arm 100 distal from a gripping section is rotatably connected to the same position of the fixed base 200, a part of the connecting section close to the gripping section of each clamp arm 100 is rotatably connected to the proximal end of the connecting rod 510 on the corresponding side, and the distal end of the connecting rod 510 is rotatably connected to the connecting seat 500 at the distal end of the drive shaft 400 by a rotation pin 610, a bolt, or the like. As the drive shaft 400 axially slides to the distal end relative to the fixed base 200, the drive shaft 400 drives the connecting rod 510 to move, and the clamp arm 100 rotates about a pin hole and opens relative to the fixed base 200 under pulling of the connecting rod 510. As the drive shaft 400 axially slides to the proximal end relative to the fixed base 200, the connecting rod 510 pulls the clamp arm 100 to rotate about the pin hole and close relative to the fixed base 200.

In some embodiments, the connecting section of the clamp arm 100 has two pairs of pin holes, each pair of pin holes overlap in the projection direction, and the pin hole close to the gripping section is hingedly connected to the connecting rod 510 by a pin, so that both the clamp arm 100 and the connecting rod 510 can rotate relatively without being separated. In some embodiments, referring to FIG. 16, the connection between the clamp arm 100 and the pin may be that the smaller end face of the pin is welded directly to the clamp arm 100, or welded by adding a loop. The pin hole away from the gripping section is a fixed hole, and a fixed pin 620 penetrates the fixed hole, so that the fixed pin 620 is fixed relative to the clamp arm 100 but can rotate about the pin hole on the fixed base 200. In some embodiments, the larger end of the fixed pin 620 is welded directly to the connecting section of the clamp arm 100.

In some embodiments, referring to FIG. 15, the connecting seat 500 is fixedly connected to the distal end of the drive shaft 400, the connecting seat 500 is symmetrically provided with two pairs of connecting holes penetrating each other, and an end of each connecting rod 510 is rotatably connected to one pair of the connecting holes. In some embodiments, a pair of pins penetrate a pair of connecting holes, so the connecting holes are not shown in the drawings. In some embodiments, the connecting seat 500 is fixedly disposed at the distal end of the drive shaft 400 by way of welding or the like, the connecting seat 500 comprises two opposite first planes and two connecting surfaces connecting the two first planes, two opposite ends of the connecting seat 500 are respectively provided with a pair of pin holes penetrating the two first planes. The pin hole is used to connect the clamp arm 100 by a pin hinge, i.e., the clamp arm 100 can rotate relatively by the hinge connection to realize opening and closing of the clamp arm 100 relative to the fixed base 200. The cross-sectional dimension of the connecting seat 500 parallel to a second plane direction decreases gradually from the proximal end to the distal end, i.e., the shape of the connecting seat 500 is any structure such as a hemispheroid, a spherical crown, or a warhead shape to more easily push the valve clamp in the body. The drive shaft 400 and the connecting seat 500 may be an integral structure or a non-integral structure. In order to ensure safety after implantation, the drive shaft 400 and the connecting seat 500 are made of a biocompatible material such as polyester, silicone, stainless steel, cobalt alloy, cobalt chromium alloy, or titanium alloy, and stainless steel or cobalt chromium alloy with high hardness are used in some embodiments.

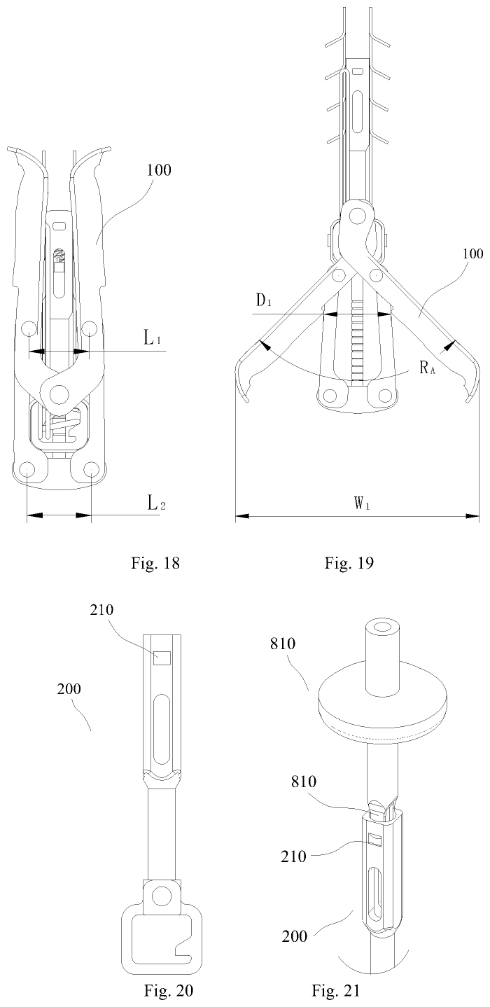

In some embodiments, referring to FIG. 18, the valve clamp is in the closed state. After the pair of clamp arms 100 close relative to the fixed base 200, the distance between the two clamp arms 100 and the center of rotation of the connecting rod 510 ranges from 2.4 mm to 4 mm. L₁ represents the distance between the two clamp arms 100 and the center of rotation of the connecting rod 510. When L₁ is too large, the outer diameter of the delivery sheath would be large, which may cause a serious wound to an intervention path; when L₁ is too small, the leaflet 700 would be clamped too tightly, which may easily cause injury to the leaflet 700. The distance between the center holes of the two pins on the connecting seat 500 is L₂, and L₂=(0.8 to 1.6)*L₁. The reason for this setting is: the difference between the distance of the two pin holes of the connecting seat 500 and the hole distance of the pins affects a closing effect of the valve clamp. The larger the difference, the wider the distance of the two pin holes of the connecting seat 500, the smaller the driving force required to close the valve clamp, and the more easily the leaflet 700 is clamped too tightly, resulting in excessive pull stress of the clamp arm 100 on the leaflet 700 which is easy to cause injury or perforation of the leaflet 700; the smaller the difference, the smaller the distance of the two pin holes of the connecting seat 500, the larger the driving force required to close the valve clamp, the larger the load required by the delivery system, and the easier it is to cause mandrel fracture or instrument failure.

In some embodiments, referring to FIG. 19, when the valve clamp opens and the pair of clamp arms 100 open to an everted state relative to the fixed base 200, the everted angle between the two clamp arms 100 ranges from 260° to 300°; the span of the ends of the two clamp arms 100 ranges from 14 mm to 18 mm. In some embodiments, after the valve clamp grips the leaflet 700, if the clamping position is found to be unreasonable or the effect is not ideal, the leaflet 700 needs to be re-captured after being released or adjusted in position or angle. When the leaflet 700 is released, the gripping arm 300 on both sides is pulled up by the control wire, and then the connecting rod 510 drives the clamp arms 100 on both sides to be everted downward, so that the leaflet 700 slips from the clamp arm 100, and the valve clamp is disengaged from wrapped chordae tendineae by inversion of the clamp arm 100.

Referring to FIG. 19, the distance between positions where the clamp arm 100 on each side abuts the connecting rod 510 on the side ranges from 3.6 mm to 4.4 mm when the pair of clamp arms 100 open to the everted state relative to the fixed base 200. While everting the clamp arm 100, the outside of the connecting rod 510 abuts the outside of the clamp arm 100, preventing the clamp arm 100 from being further everted, so an angle RA between the clamp arms 100 on the two sides after eversion is related to a distance Di between positions where the clamp arm 100 on each side abuts the connecting rod 510 on the side. The smaller the Di, the larger the angle RA between the clamp arms 100 on the two sides after eversion, the smaller a span Wi of the ends of the two clamp arms 100, the easier the operation of pulling the valve clamp back from the ventricle to the atrium, preventing the end of the clamp arm 100 from hooking the leaflet 700 or chordae tendineae. The larger the Di, the smaller the angle RA between the clamp arms 100 on the two sides after eversion, which is not easy for the leaflet 700 to slip from the inner surface of the clamp arm 100 and release. In some embodiments, in the everted state, the everted angle RA between the clamp arms 100 on the two sides ranges from 260° to 300°, and from 265° to 275° in some embodiments. The span Wi of the ends of the two clamp arms 100 on the two sides ranges from 14 mm to 18 mm. The distance Di between positions where the clamp arm 100 on each side abuts the connecting rod 510 on the side ranges from 3.6 mm to 4.4 mm.

In some embodiments, the valve clamping system further comprises a delivery component 800 comprising an outer sheath and a mandrel that is movably penetrated in the outer sheath, and the distal end of the mandrel is detachably connected to the proximal end of the drive shaft 400.

In some embodiments, referring to FIG. 20 and FIG. 21, the proximal outer wall of the fixed base 200 is symmetrically disposed with at least one detent 210 in communication with the cavity of the fixed base 200, the distal end of the outer sheath is provided with a fixing member 810 comprising two branches 811, and the end of each branch 811 is a boss. In the natural state, the two branches 811 point at the central axis of the fixing member 810. When assembled, the fixing member 810 is inserted into the fixed base 200 and the mandrel of the delivery component 800 is inserted into the outer sheath until the mandrel is inserted into the fixing member 810, the two branches 811 of the fixing member 810 are lifted outward, and the boss at the end of the branch 811 snaps into the two detents 210 of the fixed base 200, thereby connecting the fixed base 200 to the fixing member 810, i.e., connecting the valve clamp and the delivery component 800. When the mandrel is withdrawn from the fixing member and the outer sheath, the two branches 811 restore an inward natural state and are disengaged from the two detents 210 of the fixed base 200, so that the valve clamp is disconnected from the delivery component 800. The fixing member 810 is made of a material having a certain hardness and elasticity, such as nickel-titanium. The outer sheath may use a multi-layer composite tubular body. The mandrel is made of a stainless steel material.

The inside of the fixed base 200 is provided with a circular through hole as a penetrating channel of the drive shaft 400 which slidably penetrates into the penetrating channel of the fixed base 200 in the axial direction. The proximal end of the drive shaft 400 is provided with external threads for connection with the mandrel of the delivery component 800 to control axial movement of the drive shaft 400 by the mandrel. After the gripping arm 300 and the clamp arm 100 clamp the tissue of the leaflet 700, the drive shaft 400 is driven to move axially and proximally by the mandrel, the drive shaft 400 drives the connecting rod 510 to rotate, and the connecting rod 510 drives the clamp arm 100 to close relative to the fixed base 200 until the clamp arms 100 fully closes relative to the fixed base 200, which makes the clamp hang below the leaflets 700 in a closed state.

Referring again to FIG. 1, in order to effectively treat “mitral valve regurgitation” of different patients in which spacing of the leaflets 700 is different, the valve clamp is provided with an adjusting portion 900. The adjusting portion 900 is located inside the valve clamp and abuts the clamp arm 100 to adjust the pulling degree of the tissue of the leaflet 700 by the valve clamp. The adjusting portion 900 comprises a first end 901 and a second end 902 disposed opposite to the first end 901. The first end 901 is an open end, and the second end 902 is a closed end gathered by a head. The head of the second end 902 and the fixed base 200 are fixed together by a common detachable or undetachable connection means such as welding, bonding, threaded connection, crimping, bolt locking, or the like, and welding connection is used in this embodiment. The head has an inner diameter that is at least 0.01 mm, and 0.05 mm to 2 mm in some embodiments larger than the outer diameter of the fixed base 200, so that the adjusting portion 900 can be welded to the fixed base 200. The inside of the adjusting portion 900 is hollow, and the fixed base 200, the drive shaft 400, and the fixing member 810 at the distal end of the delivery component 800 are located inside the adjusting portion 900, and the mandrel and the drive shaft 400 are movable relative to the fixed base 200 along the opening of the adjusting portion.

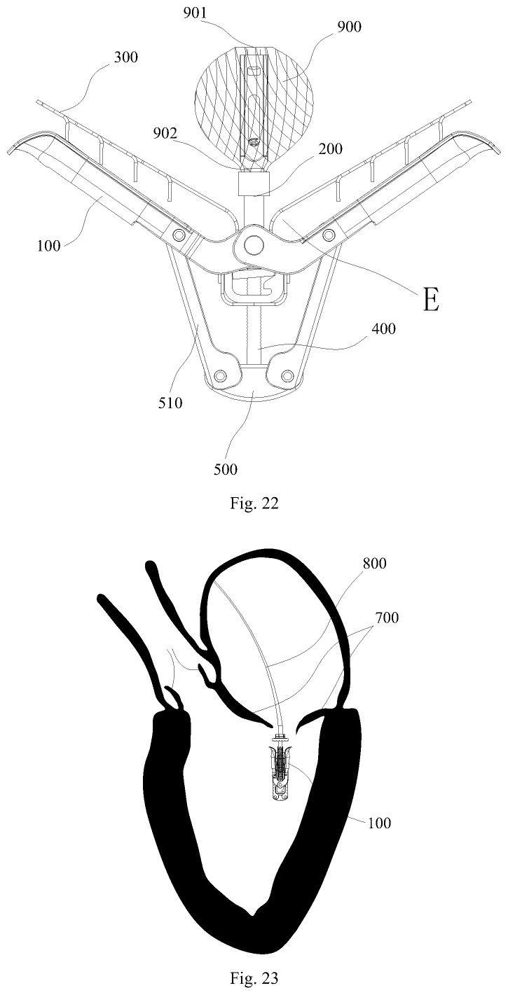

Referring to FIG. 22, the adjusting portion 900 comprises an elastic body. The adjusting portion 900 is filled between the anterior leaflet and posterior leaflet of the gripped mitral valve and abuts the clamp arm 100, which means: (1) the elastic body has a cushioning effect on the pulsing leaflets 700, thereby realizing adjustable pulling degree of the leaflets 700 by the clamp to avoid injury to the leaflets 700; (2) the elastic body can be squeezed and deformed by the pulse of the leaflets 700, the generated elastic force pushes the part of the leaflets 700 close to the elastic body in a direction away from the fixed base 200 to make the clamping angle between the anterior leaflet and the posterior leaflet of the mitral valve less than the opening angle between the clamp arms 100, which can reduce pulling of the leaflet 700 by the valve clamp, so that the pulling degree of the leaflet 700 by the clamp always remains within a reasonable range; and (3) the elastic body can cushion direct flushing of blood flow inside the valve clamp, avoid the valve clamp from falling off due to the continuous flushing of blood, and prevent blood from being deposited at a dead angle (E in FIG. 22) between the gripping portions of the valve clamp to form thrombus; and (4) when the elastic body is under pressure of the leaflet 700, the elastic body produces a degree of deformation, and the deformation degree increases with an increase in pressure, so as to avoid that the elastic body in turn acts on the clamp arm 100 under squeezing force of the clamp arm 100 after gripping the leaflet 700, thereby ensuring that the gripping effect of the leaflets 700 by the valve clamp after release remains consistent with that before release.

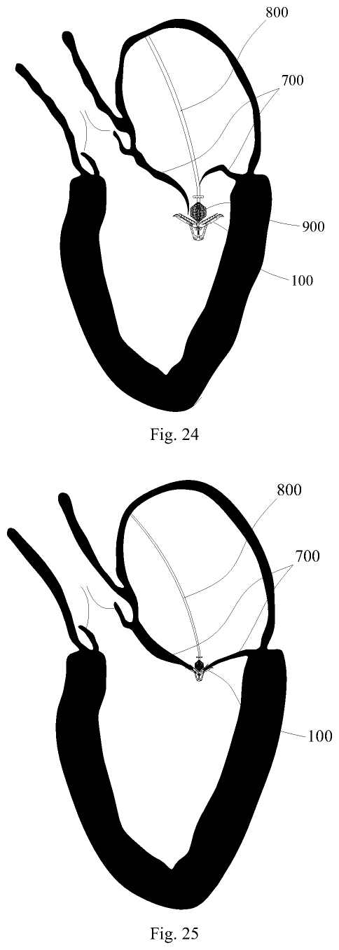

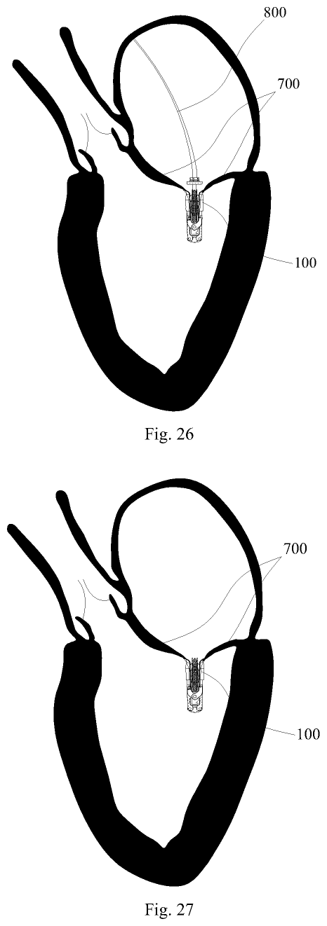

Taking transseptal approach and repair of the mitral valve as an example, a use process of a valve clamp provided in the present disclosure is described below:
S1: referring to FIG. 23, pushing the drive shaft 400 and the valve clamp connected thereto from the left atrium via the mitral valve to the left ventricle by the delivery component 800;
S2: adjusting the valve clamp to approach the leaflets 700, i.e., the anterior leaflet and the posterior leaflet of the mitral valve;
S3: referring to FIG. 24, pulling the mandrel and the drive shaft 400 proximally, driving the clamp arm 100 to open relative to the fixed base 200, and adjusting the direction of the clamp arm 100, during which a relative position of the clamp arm 100 and the anterior and posterior leaflets of the mitral valve can be observed by an X-ray device, so that the clamp arm 100 is perpendicular to a coaptation line of the mitral valve;
S4: referring to FIG. 25, withdrawing the entire valve clamp proximally, so that the clamp arm 100 holds the leaflet 700 on the left ventricle side;
S5: referring to FIG. 26, loosening the control wire to release the gripping arms 300 on both sides, wherein the gripping arm 300 on each side presses the leaflet 700 on the atrium side and fixes the leaflet 700 in cooperation with the clamp arm 100 on the side to realize complete clamping of the leaflet 700;
S6: pushing the mandrel and the drive shaft 400 distally to drive the clamp arms 100 to close when the anterior leaflet and the posterior leaflet of the mitral valve are clamped between the pair of clamp arms 100 and the gripping arms 300 respectively; and
S7: releasing the threaded connection between the mandrel and the drive shaft 400 and withdrawing the mandrel, so that the two branches 811 of the fixing member 810 restores a state of converging to the center axis, the boss is disengaged from the two detents of the fixed base 200, and the valve clamp is disconnected from the delivery component 800, and then withdrawing the delivery component 800 from the body, resulting in an implanted state as shown in FIG. 27, during which the valve clamp pulls the anterior leaflet and the posterior leaflet of the mitral valve toward each other to obtain a double orifice structure and complete edge-to-edge repair of the mitral valve.

1. A valve clamp for preventing leaflet injury, comprising a fixed base and a pair of clamp arms that are openably and closeably disposed on opposite sides of the fixed base, wherein each of the clamp arms has a connection end connected to the fixed base, a free end opposite to the connection end, and a gripping section between the connection end and the free end, the gripping section is at least partially recessed inwardly to form a capture zone, and the capture zone extends toward the free end and then extends toward a direction away from the capture zone of the other clamp arm to form a first flanging section;
wherein an anti-slipping portion is provided between the capture zone and the first flanging section, and a depth of the anti-slipping portion is less than a depth of the capture zone.

2. The valve clamp for preventing leaflet injury of claim 1, wherein an included angle A ranging from 90° to 150° is formed between a tangent of an end of the first flanging section and an axial direction of the gripping section.

3. The valve clamp for preventing leaflet injury of claim 2, wherein an included angle B ranging from 20° to 60° is formed between axial directions of the two gripping sections after the pair of clamp arms close relative to the fixed base.

4. The valve clamp for preventing leaflet injury of claim 3, wherein an included angle C ranging from −30° to 10° is formed between an extending line of the first flanging section and a line perpendicular to a central axis of the valve clamp.

5. The valve clamp for preventing leaflet injury of claim 4, wherein a half of the angle B plus 90° is equal to a sum of the angle A and the angle C.

6. (canceled)

7. The valve clamp for preventing leaflet injury of claim 1, wherein the clamp arm further comprises a pair of second flanging sections, and each of the second flanging sections is formed by extending from the capture zone to each side and is smoothly connected to each side of the first flanging section.

8. The valve clamp for preventing leaflet injury of claim 7, wherein the clamp arm further comprises a pair of third flanging sections, and
each of the third flanging sections is formed by extending outward from each side of the capture zone and is smoothly connected to the second flanging section on the same side thereof.

9. The valve clamp for preventing leaflet injury of claim 8, wherein the third flanging section sequentially comprises the following from a distal end to a proximal end: a first part; a second part; and a third part, wherein a transverse dimension of the first part gradually increases from the distal end toward the proximal end to be substantially equal to a transverse dimension of the second part, the transverse dimension of the second part decreases gradually from the distal end toward the proximal end to be substantially equal to a transverse dimension of the third part.

10. The valve clamp for preventing leaflet injury of claim 8, wherein the clamp arm further comprises a pair of fourth flanging sections, and each of the fourth flanging sections is formed by extending proximally from the third flanging section on the same side thereof respectively.

11. The valve clamp for preventing leaflet injury of claim 10, wherein the clamp arm is provided with a hole for assembly, the fourth flanging section is provided next to the hole, and the fourth flanging section has an axial dimension larger than a diameter of the hole.

12. The valve clamp for preventing leaflet injury of claim 1, further comprising a pair of gripping arms, wherein each of the gripping arms is disposed between the fixed base and the clamp arm, and the gripping arm is at least partially received in the inner wall of the capture zone in a natural state.

13. A valve clamping system comprising the valve clamp for preventing leaflet injury of claim 1 and a drive component comprising:
a drive shaft penetrating the fixed base;
a connecting seat disposed at a distal end of the drive shaft; and
a pair of connecting rods pivotably connected to both sides of the connecting seat,
wherein an end of the connecting rod is connected to the clamp arm, and
the drive shaft slides axially relative to the fixed base to drive the clamp arm to open and close relative to the fixed base by rotation of the connecting rod.

14. The valve clamping system of claim 13, wherein the connecting seat is fixedly connected to the distal end of the drive shaft, the connecting seat is symmetrically provided with two pairs of connecting holes penetrating each other, and an end of each of the connecting rods is rotatably connected to one pair of the connecting holes.

15. The valve clamping system of claim 13, wherein after the pair of clamp arms close relative to the fixed base, a distance between the two clamp arms and a center of rotation of the connecting rod ranges from 2.4 mm to 4 mm.

16. The valve clamping system of claim 13, wherein when the pair of clamp arms open to an everted state relative to the fixed base, an everted angle between the two clamp arms ranges from 260° to 300°; and a span of ends of the two clamp arms ranges from 14 mm to 18 mm.

17. The valve clamping system of claim 13, wherein when the pair of clamp arms open to an everted state relative to the fixed base, a distance between positions where each of the clamp arms abuts the connecting rod ranges from 3.6 mm to 4.4 mm.

18. The valve clamping system of claim 13, further comprising a delivery component that comprises an outer sheath and a mandrel movably penetrated in the outer sheath, and a distal end of the mandrel is detachably connected to a proximal end of the drive shaft.

19. A method for repairing a valve, comprising:
pushing a drive shaft and the valve clamp of claim 1 connected thereto from an atrium of a subject in need thereof via a valve to a ventricle by a delivery component;
adjusting the valve clamp to approach leaflets, i.e., an anterior leaflet and a posterior leaflet of the valve;
pulling a mandrel and the drive shaft proximally, driving the clamp arm to open relative to the fixed base, and adjusting the direction of the clamp arm, so that the clamp arm is perpendicular to a coaptation line of the valve;
withdrawing the entire valve clamp proximally, so that the clamp arm holds the leaflet on the ventricle side;
loosening a control wire to release the gripping arms on both sides, wherein the gripping arm on each side presses the leaflet on the atrium side and fixes the leaflet in cooperation with the clamp arm on the side to realize complete clamping of the leaflet;
pushing the mandrel and the drive shaft distally to drive the clamp arms to close when the anterior leaflet and the posterior leaflet of the valve are clamped between the pair of clamp arms and gripping arms respectively; and
releasing connection between the mandrel and the drive shaft and withdrawing the mandrel, so that the valve clamp is disconnected from the delivery component, and then withdrawing the delivery component from a body, during which the valve clamp pulls the anterior leaflet and the posterior leaflet of the valve toward each other to obtain a double orifice structure and complete edge-to-edge repair of the valve.

20. The method of claim 19, wherein the subject is a human.

21. The method of claim 19 or 20, wherein the valve is selected from the group consisting of mitral valve and tricuspid valve.
